(19) United States
(12) Patent Application Publication
(10) Pub. No.: US 2025/0275898 A1
(43) Pub. Date: Sep. 4, 2025

(54) MIXTURE OF STABILIZED STANNOUS IONS COMPLEX FOR HIGH-WATER CONTENT ORAL CARE PRODUCT

(71) Applicant: Honeywell International Inc., Charlotte, NC (US)

(72) Inventors: Junjia Fang, Suzhou (CN); Siyue Li, Hong Kong (CN); Yanyan Song, Suzhou (CN); Xi Cui, Suzhou (CN)

(21) Appl. No.: 19/063,009

(22) Filed: Feb. 25, 2025

Related U.S. Application Data
(60) Provisional application No. 63/560,903, filed on Mar. 4, 2024.

(51) Int. Cl. A61K 8/18; A61K 8/06; A61K 8/44; A61K 8/60; A61K 8/67; A61K 8/86; A61K 8/92; A61Q 11/00

(52) U.S. Cl. CPC A61K 8/18; A61K 8/064; A61K 8/066; A61K 8/44; A61K 8/602; A61K 8/676; A61K 8/678; A61K 8/86; A61K 8/922; A61Q 11/00

(57) ABSTRACT

A stable stannous ion emulsion complex formed from combining a first aqueous phase with an oil phase to form a water in oil emulsion; forming a second aqueous phase; and combining the second aqueous phase with the water in oil emulsion. The first aqueous phase including water, stannous ions, a chelating agent, and a pH buffer. The oil phase including an oil and a first emulsifier. The water in oil emulsion including equal amounts of the oil phase and the first aqueous phase. The second aqueous phase including water, stannous ions, a chelating agent, a pH buffer, and a second emulsifier.

### CROSS-REFERENCE

This application claims the benefit under 35 U.S.C. § 119 (e) of U.S. Provisional Application No. 63/560,903 entitled “A MIXTURE OF STABILIZED STANNOUS IONS COMPLEX FOR HIGH-WATER CONTENT ORAL CARE PRODUCT”, filed on Mar. 4, 2024, the entire disclosure of which is incorporated by reference in its entirety.

### FIELD

The present disclosure relates generally a composition for an oral care product comprising stabilized stannous ion complex with a high-water content.

### BACKGROUND

Fluorides have demonstrated to be an effective ingredient in reducing the occurrence of dental caries. Thus, fluorinated compounds, such as sodium fluoride and sodium fluorophosphate, are often used in oral care products. Stannous ions have been proved to have multiple preferential effects in oral care products, such as anti-plaque, anti-caries, and anti-allergy effects, since the mid-late 1950s. However, stannous ions are easily dissociated in a dentifrice formulation and thus engages in complex chemical changes including hydrolysis, chelation, oxidation, and precipitation. As a result, the preferential effects are deteriorated. Low-water or anhydrous formulations are effective for stannous ions, but often experience a series of issues, including poor esthetics, attenuated foam, and weakened flavor impact.

For these reasons, a stable stannous ions complex in a high-water system without the use of stannous chloride is needed.

### SUMMARY

The present disclosure provides a method of forming a stable stannous ion complex or an oral care product, including forming a first aqueous phase by mixing water, stannous ions, and a chelating agent; forming an oil phase by mixing an oil and a first emulsifier; combining equal parts of the first aqueous phase and the oil phase to form water in oil (W/O) emulsion; forming a second aqueous phase by mixing water, stannous ions, and a chelating agent; and combining the water in oil emulsion and the second aqueous phase to form a stannous ion emulsion complex. A mass ratio of the oil to the first emulsifier is 1:1 to 1:20. A mass ratio of the water in oil emulsion to the second aqueous phase is 1:1 to 1:10.

The present disclosure further provides an oral care product containing stannous ions, including: forming a first aqueous phase by mixing water, stannous ions, a chelating agent, and a pH buffer; forming an oil phase by mixing an oil and a first emulsifier; combining equal parts of the first aqueous phase and the oil phase to form a water in oil (W/O) emulsion; forming a second aqueous phase by mixing water, stannous ions, a chelating agent, a pH buffer, and a second emulsifier; combining the water in oil emulsion and the second aqueous phase to form a stannous ion emulsion complex; dispersing the stannous ion emulsion complex in a solution to form a W/O/W stannous ion composition; and combining the W/O/W stannous ion composition with an oral care product. A ratio of the one oil to the first emulsifier is 1:1 to 1:20. A ratio of the water in oil emulsion to the second aqueous phase is 1:1 to 1:10.

### BRIEF DESCRIPTION OF THE DRAWINGS

The above-mentioned and other features and advantages of this disclosure, and the manner of obtaining them, will become more apparent, and will be better understood by reference to the following description of the exemplary embodiments taken in conjunction with the accompanying drawings, wherein:

FIG. 1 is a flow chart of a method of forming a stable stannous ion complex or an oral care product of the present disclosure.

Corresponding reference characters indicate corresponding parts throughout the several views. Although the drawings represent embodiments of various features and components according to the present disclosure, the drawings are not necessarily to scale and certain features may be exaggerated in order to better illustrate and explain the present disclosure. The exemplification set out herein illustrates an embodiment of the invention, and such an exemplification is not to be construed as limiting the scope of the invention in any manner.

### DETAILED DESCRIPTION

For the purposes of promoting an understanding of the principles of the present disclosure, reference is now made to the embodiments illustrated in the drawings, which are described below. The exemplary embodiments disclosed herein are not intended to be exhaustive or to limit the disclosure to the precise form disclosed in the following detailed description. Rather, these exemplary embodiments were chosen and described so that others skilled in the art may utilize their teachings.

The present disclosure provides a stable emulsified stannous ions emulsion complex for high-water content systems such as toothpaste and mouthwash.

### I. Stannous Ion Emulsion Complex Composition

Known high water formulations typically stabilize stannous ions by adding stannous chloride (SnCl₂) and chelating agents, such as phosphoric acid, carboxylic acid, and their salts. Chelating agents are added to stabilize the tin ions and slow down the oxidation rate in order to maintain the efficacy of stannous ions. However, such an anhydrous formula has a poor user experience due to poor consistency and yellowing of the teeth due to excessive addition of stannous ions.

The stannous ion emulsion complex composition may comprise at least one oil, a first emulsifier, a second emulsifier, water, chelating agents, pH buffer solution, stannous ion, and, optionally, at least one additive. Suitable oils may include flavor oil, such as α-terpineol, eugenol, limonene, citral, menthol, geraniol, linalyl acetate, vitamin E, and vitamin C. Examples of suitable long chain triglyceride-containing oils include almond oil, babassu oil, borage oil, black currant seed oil, canola oil, caster oil, corn oil, cottonseed oil, olive oil, palm oil, peanut oil, safflower oil, seasame oil, linseed oil, grape oil, coconut oil, soybean oil, cocoa butter, vitamin E oil and other suitable essential oils or their combinations.

Suitable emulsifiers may comprise span 20, span 40, span 60, span 80, tween 20, tween 40, tween 60, tween 80, PEG-20, PEG-40, PEG-60, PEG-80, PEG-400, AEO-3, AEO-7, AEO-9 or their combinations.

Suitable chelating agents may comprise glutamic acid diacetate (GLDA), aspartic acid N, N diacetic acid (ASDA), GLDA/ASDA salts, and combinations thereof.

### II. Method of Preparing Stannous Ion Emulsion

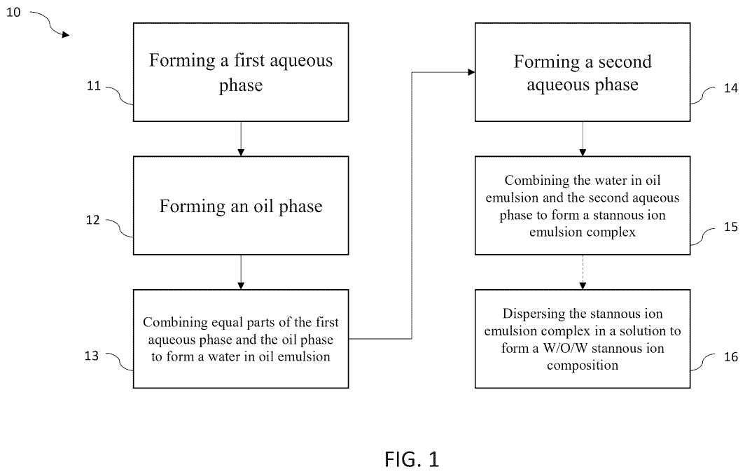

The present disclosure provides a method 10 of forming a stable stannous ion complex or an oral care product, as shown in FIG. 1. The stabilized stannous ion emulsion complex of the present disclosure may be formed by combining an oil phase and a first aqueous phase, creating a water in oil (W/O) emulsion, with a second aqueous phase.

### A. Preparation of the First Aqueous Phase

In step 11 of method 10, a first aqueous phase is formed. To prepare the first aqueous phase, water, stannous ions, a chelating agent, and a pH buffer may be mixed together. The first aqueous phase may comprise an amount of water from 5 wt. %, 25 wt. %, or 50 wt. % to 75 wt. %, 80 wt. %, or 90 wt. %, or any range using any two of the forgoing values as endpoints, such as 5 wt. % to 90 wt. %, 25 wt. % to 80 wt. %, or 50 wt. % to 75 wt. %, based on the total weight of the first aqueous phase.

The first aqueous phase may comprise an amount of stannous ions from 5 wt. %, 10 wt. %, or 15 wt. % to 20 wt. %, 25 wt. %, or 30 wt. %, or any range using any two of the forgoing values as endpoints, such as 5 wt. % to 30 wt. %, 10 wt. % to 25 wt. %, or 15 wt. % to 20 wt. %, based on the total weight of the first aqueous phase.

The first aqueous phase may comprise an amount of chelating agent from 5 wt. %, 15 wt. %, or 25 wt. % to 35 wt. %, 45 wt. %, or 60 wt. %, or any range using any two of the forgoing values as endpoints, such as 5 wt. % to 60 wt. %, 15 wt. % to 45 wt. %, or 25 wt. % to 35 wt. %, based on the total weight of the first aqueous phase.

The first aqueous phase may comprise an amount of pH buffer from 0 wt. %, 0.5 wt. %, or 1.0 wt. % to 1.5 wt. %, 2.0 wt. %, or 5 wt. %, or any range using any two of the forgoing values as endpoints, such as 0 wt. % to 5 wt. %, 0.5 wt. % to 2.0 wt. %, or 1.0 wt. % to 1.5 wt. %, based on the total weight of the first aqueous phase.

### B. Preparation of the Oil Phase

Method 10 further comprises forming an oil phase 12. The oil phase may be prepared by mixing oil and a first emulsifier together. The oil phase may comprise an amount of oil from 50 wt. %, 60 wt. %, 70 wt. % to 80 wt. %, 90 wt. %, or 95 wt. %, or any range using any two of the forgoing values as endpoints, such as 50 wt. % to 95 wt. %, 60 wt. % to 90 wt. %, or 70 wt. % to 80 wt. %, based on the total weight of the oil phase.

The oil phase may comprise an amount of the first emulsifier from 5 wt. %, 15 wt. %, or 25 wt. % to 35 wt. %, 45 wt. %, or 50 wt. %, or any range using any two of the forgoing values as endpoints, such as 5 wt. % to 50 wt. %, 15 wt. % to 45 wt. %, or 25 wt. % to 35 wt. %, based on the total weight of the oil phase.

The oil phase may comprise a mass ratio of the first emulsifier to the oil from 1:1, 1:5, or 1:7 to 1:10, 1:15, or 1:20, or any range using any two of the foregoing ratios as endpoints, such as 1:1 to 1:20, 1:5 to 1:15, or 1:7 to 1:10.

### C. Preparation of the Water in Oil (W/O) Emulsion

Once the oil phase and the first aqueous phase are formed, a water in oil emulsion may be prepared by mixing equal parts of the oil phase and the first aqueous phase (50 wt. % oil phase and 50 wt. % first aqueous phase, based on the total weight of the W/O emulsion) together, as seen in step 13 of method 10.

### D. Preparation of the Second Aqueous Phase

In step 14, a second aqueous phase may be formed. To prepare the second aqueous phase, water, stannous ions, a chelating agent, pH buffer, and a second emulsifier may be combined. The second aqueous phase may comprise similar amounts of water, stannous ions, a chelating agent, and pH buffer as the first aqueous phase.

The second aqueous phase may comprise an amount of a second emulsifier from 0 wt. %, 1 wt. %, or 5 wt. % to 10 wt. %, 15 wt. %, or 20 wt. %, or any range using any two of the forgoing values as endpoints, such as 0 wt. % to 20 wt. %, 1 wt. % to 15 wt. %, or 5 wt. % to 10 wt. %, based on the total weight of the second aqueous phase.

### E. Preparation of the Stannous Ion Emulsion Complex

The second aqueous phase and the W/O emulsion are combined to form a stannous ion emulsion complex in step 15. The stannous ion emulsion complex may comprise an amount of W/O emulsion from 10 wt. %, 20 wt. %, or 30 wt. % to 35 wt. %, 40 wt. %, or 50 wt. %, or any range using any two of the forgoing values as endpoints, such as 10 wt. % to 50 wt. %, 20 wt. % to 40 wt. %, or 30 wt. % to 35 wt. %, based on the total weight of the stannous ion emulsion complex.

The stannous ion emulsion complex may comprise an amount of second aqueous phase from 50 wt. %, 60 wt. %, or 65 wt. % to 70 wt. %, 80 wt. %, or 90 wt. %, or any range using any two of the forgoing values as endpoints, such as 50 wt. % to 90 wt. %, 60 wt. % to 80 wt. %, or 65 wt. % to 70 wt. %, based on the total weight of the stannous ion emulsion complex.

The stannous ion emulsion complex may comprise a mass ratio of W/O emulsion to second aqueous phase from 1:1, 1:2, or 1:4 to 1:6, 1:8, or 1:10, or any range using any two of the foregoing ratios as endpoints, such as 1:1 to 1:10, 1:2 to 1:8, or 1:4 to 1:6.

Optionally, in step 16, once the stannous ion emulsion complex is created, it may be re-dispersed and homogenized into solution to prepare a stabilized water in oil, in water (W/O/W) stannous ion composition.

### II. Properties of Stannous Ion Emulsion

To test the stability of the stannous ion emulsion complex in solution, an iodine titration for retention testing of tin ions is performed.

The iodine titration method used to evaluate Sn²⁺ content may use a Mettler Toledo T50 potentiometric titrator and a platinum electrode. The electrode calibration can be found in the instruction manual. Sodium thiosulfate solution was used as a reductive agent and iodine solution was used as an oxidized agent.

The stannous ion emulsion complex is then put into the oven at 50° C. and 75% humidity for a speed up aging process to evaluate the stability. If the stannous ion emulsion complex has a Sn²⁺ retention above 90%, the emulsion complex has a good stability.

TABLE A
Range of Stability of Stannous Ion Emulsion Complex

| | 1 Day | 7 Days | 14 Days | 30 Days |
|---|---|---|---|---|
| Sn²⁺ wt. % | 9.26 | 9.19 | 9.14 | 8.85 |
| pH | 6.49 | 6.27 | 6.25 | 6.33 |

The W/O/W stannous ion composition may provide an antibacterial properties when included in an oral care product, such as toothpaste or mouthwash. The efficacy of the antibacterial properties of the W/O/W stannous ion composition in an oral care product may be tested according to WS/T 650-2019.

The following method was used to determine the antibacterial properties of the W/O/W stannous ion composition according to WS/T 650-2019.

To a sterile test tube, 5.0 mL of Stannous ion complex is added. The test tube is placed in a 20° C.±1° C. water bath for 5 minutes. Then, 0.1 mL of test bacterial suspension is added and mixed quickly and timed immediately.

Acinetobacter baumannii and Streptococcus mutans were selected as test bacteria. The test bacteria and antibacterial agent interact with each other for 30 s, and 0.5 mL of the test bacteria and antibacterial agent mixture is taken and added to 4.5 mL of the neutralizing agent.

After 10 minutes of neutralization with a neutralizing agent, 1.0 mL of the mixed solution of the test bacteria and antibacterial agent is taken from each tube. The number of viable bacteria is measured using the live bacterial culture counting method. Two petri dishes are inoculated into each tube of the sample solution. When there are a large number of colonies growing on the plate, PBS can be used for 10 times series dilution, and then live bacterial culture counting can be carried out. Simultaneously, PBS may be used instead of disinfectant for parallel testing as a positive control. The positive control recovered a colony count of 1.0×104CFU/mL˜9.0×104CFU/mL. The same batch of diluent, neutralizer, and culture medium may be used as negative controls.

All experimental and control samples are cultured at 36° C.±1° C., and the final results are observed after 48 hours of bacterial growth. Candida albicans needs to be cultured for 72 hours to observe the final results. The experiment is repeated three times and then the sterilization rate may be calculated.

If the sterilization rate is calculated to be ≥90%, the composition is judged to have antibacterial effect. The sterilization rate specified is calculated to be ≥99%, the composition is judged to have a strong antibacterial effect.

The W/O/W stannous ion composition in an oral care product may have an antibacterial efficacy of at least 90%, at least 92%, at least 94%, at least 96%, at least 98%, or at least 99% as determined by WS/T 650-2019, as described above.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practices in the art to which this invention pertains.

### Examples

### Example 1: Formulation of the Stannous Ion Emulsion Complex Composition 1

### First Aqueous Phase

The first aqueous phase was formulated according to Table 1 and the method described above.

TABLE 1
First Aqueous Phase Formulation

| Component | Amount (wt. %) |
|---|---|
| Water | 69 |
| Stannous Ions | 10 |
| ASDA-4Na | 20 |
| pH Buffer Solution | 1 |

### Oil Phase

The oil was formulated according to Table 2 and the method described above.

TABLE 2
Oil Phase Formulation

| Component | Amount (wt. %) |
|---|---|
| Oil | 90 |
| Span 60 | 10 |

### Water in Oil (W/O) Emulsion

The W/O emulsion was formulated according to Table 3 using the oil phase from Table 2 and the first aqueous phase from Table 1 and the method described above.

TABLE 3
Water in Oil (W/O) Emulsion Formulation

| Component | Amount (wt. %) |
|---|---|
| Oil phase | 50 |
| First aqueous phase | 50 |

### Second Aqueous Phase

The second aqueous phase was formulated according to Table 4 and the method described above.

TABLE 4
Second Aqueous Phase Formulation

| Component | Amount (wt. %) |
|---|---|
| Water | 68.5 |
| Stannous Ions | 10 |
| ASDA-4Na | 20 |
| pH Buffer Solution | 1 |
| PEG-40 | 0.5 |

### Stannous Ion Emulsion Complex

The stannous ion emulsion complex was formulated according to Table 5 using the W/O emulsion of Table 3 and second aqueous phase of table 4 and the method described above. The stannous ion emulsion complex may then be re-dispersed and homogenized to obtain a stable W/O/W stannous ion composition.

TABLE 5
Stannous Ion Emulsion Complex Composition

| Component | Amount (wt. %) |
|---|---|
| W/O emulsion | 20 |
| Second aqueous phase | 80 |

### Example 2: Formulation of the Stannous Ion Emulsion Complex Composition 2

### First Aqueous Phase

The first aqueous phase was formulated according to Table 6 and the method described above.

TABLE 6
First Aqueous Phase Formulation

| Component | Amount (wt. %) |
|---|---|
| Water | 50 |
| Stannous ions | 20 |
| GLDA-4Na | 30 |

### Oil Phase

The oil was formulated according to Table 7 and the method described above.

TABLE 7
Oil Phase Formulation

| Component | Amount (wt. %) |
|---|---|
| Oil | 70 |
| AEO-5 | 30 |

### Water in Oil (W/O) Emulsion

The W/O emulsion was formulated according to Table 8 using the oil phase from Table 7 and the first aqueous phase from Table 6 and the method described above.

TABLE 8
Water in Oil Emulsion Formulation

| Component | Amount (wt. %) |
|---|---|
| Oil phase | 80 |
| First aqueous phase | 20 |

### Second Aqueous Phase

The second aqueous phase was formulated according to Table 9 and the method described above.

TABLE 9
Second Aqueous Phase Formulation

| Component | Amount (wt. %) |
|---|---|
| Water | 39.5 |
| Stannous ion | 30 |
| GLDA-4Na | 30 |
| Tween-80 | 0.5 |

### Stannous Ion Emulsion Complex

The stannous ion emulsion complex was formulated according to Table 10 using the W/O emulsion of Table 8 and second aqueous phase of table 9 and the method described above. The stannous ion emulsion complex may then be re-dispersed and homogenized to obtain a stable W/O/W stannous ion composition.

TABLE 10
Stannous ion Emulsion Complex Composition

| Component | Amount (wt. %) |
|---|---|
| W/O emulsion | 50 |
| Second aqueous phase | 50 |

### Example 3: Properties of the Stannous Ion Emulsion Complex Composition

In this example, W/O/W Stannous ions compositions were prepared by re-dispersing and homogenizing the stannous ions emulsion complex composition described in Table 11. The stability test of stannous ions emulsion complex composition is indicated in Table 6. The results showed that Stannous ions emulsion complex have a retention above 90% during 50° C. aging test. And the pH of W/O/W emulsion complex is relatively stable.

TABLE 11
Stability Test Results of Stannous Ion Emulsion
Complex Stock Solution at 50° C.

| | 1 | 7 | 14 | 30 |
|---|---|---|---|---|
| | day | days | days | days |
| Stannous ion % | 9.26 | 9.19 | 9.14 | 8.85 |
| pH | 6.49 | 6.27 | 6.25 | 6.33 |

### Example 4: Stannous Ion Emulsion Complex in Mouthwash

In this Example, the stannous ions emulsion complex formulated in Table 5 was re-dispersed and homogenized to obtain a stable W/O/W stannous ions composition. The stability of the mouthwash prepared by W/O/W Stannous ions compositions was tested in 50° C. The results were shown in Table 12 and Table 13, indicated that the mixture could further enhance Sn²⁺ and stabilization performance and retain efficacy. There is a synergy effect of chelation and emulsification for providing a stable Stannous ions emulsion complex mixture with readiness to use in oral care product (toothpaste and mouthwash) formulation especially for high-water content oral care product. The stable W/O/W Stannous ions composition was used in a mouthwash composition prepared according to Table 8.

TABLE 12
Stability Test Results of Stannous Ion
Emulsion Complex Mouthwash at 50° C.

| | | Sn²⁺ | Sn²⁺ | Sn²⁺ |
|---|---|---|---|---|
| | | wt. % | wt. % | wt. % |
| | Oil Phase | (7 d) | (22 d) | (32 d) |
| Emulsified | α-terpineol | 82.91% | 55.43% | 21.92% |
| stabilized | W/O/W | | | |
| Stannous ion | Eugenol | 94.77% | 70.88% | 53.76% |
| complex (pH | W/O/W | | | |
| 6.5) | Aqueous phase only | 87.73% | 62.30% | 31.09% |
| | (control) | | | |
| Emulsified | α-terpineol | 90.32% | 71.45% | 48.99% |
| stabilized | W/O/W | | | |
| Stannous ion | Eugenol | 87.43% | 73.01% | 52.79% |
| complex (pH | W/O/W | | | |
| 7.0) | Aqueous phase only | 88.18% | 58.04% | 22.05% |
| | (control) | | | |

TABLE 13
Stannous Ion Emulsion Complex Mouthwash Composition

| Component | Amount (wt. %) |
|---|---|
| Water | 85.7 |
| Glycerol | 5 |
| Sorbitol | 5 |
| W/O/W stannous ion | 4 |
| composition | |
| Saccharin sodium | 0.1 |
| Sodium benzoate | 0.1 |
| Essence | 0.1 |

An antibacterial efficacy test was performed on mouthwash comprising the stannous ion emulsion complex at two different weight percent amounts and a mouthwash that did not comprise stannous ions. The results showed that the stannous ion emulsion complex mouthwash had better antibacterial effects than the mouthwash without stannous ions, as shown in Table 14.

TABLE 14
Efficacy Test Results on Stannous Ion
Emulsion Complex Mouthwash Formulation

| | pH | | | Type of | |
|---|---|---|---|---|---|
| Sample | value | Sn²⁺% | Condition | Bacteria | Efficacy |
| Blank | 6.04 | 0 | Contact 30 s | Streptococcus | 19.1% |
| Mouthwash | | | | mutans | |
| Mouthwash | 6.33 | 0.2699 | Contact 30 s | Streptococcus | 98.4% |
| with stannous | | | | mutans | |
| ion (~0.2 wt. | | | | | |
| %) | | | | | |
| Mouthwash | 6.22 | 0.4529 | Contact 30 s | Streptococcus | 99.3% |
| with stannous | | | | mutans | |
| ion (~0.5 wt. | | | | | |
| %) | | | | | |

### Example 4: Stannous Ion Emulsion Complex in Toothpaste

In this Example, the stannous ion emulsion complex formulated in Table 15 was re-dispersed and homogenized to obtain a stable W/OW stannous ion composition. The stable W/O/W stannous ion composition was used in a toothpaste composition prepared according to Table 9.

TABLE 15
Stannous Ion Emulsion Complex Toothpaste Composition

| Component | Amount (wt. %) |
|---|---|
| Silica | 20 |
| Glycerol | 6 |
| Sorbitol | 38 |
| W/O/W stannous ion | 8 |
| composition | |
| Xanthan Gum | 2.8 |
| Saccharin Sodium | 0.1 |
| K12 | 2 |
| pH Buffer | 2 |
| Essence | 0.1 |
| Water | 21 |

An efficacy tests was performed on toothpaste comprising the stannous ion emulsion complex and a toothpaste that did not comprise stannous ions. The results showed that the toothpaste with the stannous ion emulsion complex had strong antibacterial effect on testing bacterial, as seen in Table 16.

TABLE 16
Efficacy Test Results on Stannous Ion
Emulsion Complex Toothpaste Formulation

| | pH | | | Type of | |
|---|---|---|---|---|---|
| Sample | value | Sn²⁺% | Condition | Bacteria | Efficacy |
| Blank | 7.02 | 0 | Contact 30s | Streptococcus | >99.9 |
| Toothpaste | | | | mutans | |
| Toothpaste | 7.00 | 0.255 | Contact 30s | Streptococcus | >99.9 |
| with stannous | | | | mutans | |
| ion emulsion | | | | | |
| complex | | | | | |
| (~0.2 wt. %) | | | | | |

### ASPECTS

Aspect 1 is a method of forming a stable stannous ion complex or an oral care product, comprising: forming a first aqueous phase by mixing water, stannous ions, and a chelating agent; forming an oil phase by mixing an oil and a first emulsifier; wherein a mass ratio of the oil to the first emulsifier is 1:1 to 1:20; combining equal parts of the first aqueous phase and the oil phase to form water in oil (W/O) emulsion; forming a second aqueous phase by mixing water, stannous ions, and a chelating agent; and combining the water in oil emulsion and the second aqueous phase to form a stannous ion emulsion complex; wherein a mass ratio of the water in oil emulsion to the second aqueous phase is 1:1 to 1:10.
Aspect 2 is the method of Aspect 1, wherein the first aqueous phase comprises: 5-90 wt. % of water; 5-30 wt. % of stannous ions; and 5-60 wt. % of the chelating agent, based on a total weight of the first aqueous phase.
Aspect 3 is the method of either Aspect 1 or Aspect 2, wherein the first aqueous phase further comprises less than 2.5 wt. % of a pH buffer, based on a total weight of the first aqueous phase.
Aspect 4 is the method of any one of Aspects 1-3, wherein the oil phase comprises: 50-95 wt. % the oil; and 5-50 wt. % of the first emulsifier, based on a total weight of the oil phase.
Aspect 5 is the method of any one of Aspects 1-4, wherein the second aqueous phase comprises: 5-90 wt. % water; 5-30 wt. % of stannous ions; and 5-60 wt. % of the chelating agent, based on a total weight of the second aqueous phase.
Aspect 6 is the method of any one of Aspects 1-5, wherein the second aqueous phase further comprises less than 5 wt. % of a pH buffer; and less than 20 wt. % of a second emulsifier, based on a total weight of the second aqueous phase.
Aspect 7 is the method of any one of Aspects 1-6, wherein the stannous ion emulsion complex comprises: 10-50 wt. % of the W/O emulsion; and 50-90 wt. % of the second aqueous phase, based on a total weight of the stannous ion emulsion complex.
Aspect 8 is the method of any one of Aspects 1-7, wherein the stannous ions are one of stannous fluoride, stannous chloride dihydrate, stannous acetate, stannous gluconate, stannous oxalate, stannous sulfate, stannous lactate, stannous tartrate, and combinations thereof.
Aspect 9 is the method of any one of Aspects 1-8, wherein the chelating agent is one of GLDA, ASDA, GLDA-4Na, ASDA-4Na, and combinations thereof.
Aspect 10 is the method of any one of Aspects 1-9, wherein the first emulsifier is one of span-60, span 80, AEO-3, AEO-5, AEO-7, PEG-20, PEG-40, PEG-60, PEG-80, PEG-400, tween-20, tween-60, tween-80, and combinations thereof.
Aspect 11 is the method of any ones of Aspects 6-10, wherein the second emulsifier is one of span-60 span 80, AEO-3, AEO-5, AEO-7, PEG-20, PEG-40, PEG-60, PEG-80, PEG-400, tween-20, tween-60, tween-80, and combinations thereof.
Aspect 12 is the method of any one of Aspects 1-11, wherein the oil is at least one of α-terpineol, Eugenol, plant oil including coconut oil, soybean oil, corn oil, olive oil vitamin E oil, vitamin C, and combinations thereof.
Aspect 13 is the method of any one of Aspects 1-12, further comprising dispersing the stannous ion emulsion complex in a solution to form a W/O/W stannous ion composition.
Aspect 14 is the method of Aspect 13, wherein the W/O/W stannous ion composition has a Sn²⁺ stability of above 90% after a 1-month aging test in a 50° C. oven as determine by iodine titration testing.
Aspect 15 is a method of preparing an oral care product containing stannous ions, comprising: forming a first aqueous phase by mixing water, stannous ions, a chelating agent, and a pH buffer; forming an oil phase by mixing an oil and a first emulsifier; wherein a ratio of the one oil to the first emulsifier is 1:1 to 1:20; combining equal parts of the first aqueous phase and the oil phase to form a water in oil (W/O) emulsion; forming a second aqueous phase by mixing water, stannous ions, a chelating agent, a pH buffer, and a second emulsifier; combining the water in oil emulsion and the second aqueous phase to form a stannous ion emulsion complex; wherein a ratio of the water in oil emulsion to the second aqueous phase is 1:1 to 1:10; dispersing the stannous ion emulsion complex in a solution to form a W/O/W stannous ion composition; and combining the W/O/W stannous ion composition with an oral care product.
Aspect 16 is the method of Aspect 15, wherein the oral care product is one of toothpaste and mouthwash.
Aspect 17 is the method of either Aspect 15 or Aspect 16, wherein the oral care product has an antibacterial efficacy of at least 90-99% as determined by WS/T 650-2019 antibacterial test.
Aspect 18 is the method of any one of Aspects 15-17, wherein the first aqueous phase comprises: 5-90 wt. % of water; 5-30 wt. % of stannous ions; 5-60 wt. % of the chelating agent; and less than 5 wt. % of pH buffer, based on the total weight of the first aqueous phase.
Aspect 19 is the method of any one of Aspects 15-18, wherein the oil phase comprises: 50-95 wt. % the oil; and 5-50 wt. % of the first emulsifier, based on the total weight of the oil phase.
Aspect 20 is the method of any one of Aspects 15-19, wherein the second aqueous phase comprises: 15-90 wt. % of water; 5-20 wt. % of stannous ions; 5-40 wt. % of the chelating agent; less than 5 wt. % of pH buffer; and less than 20 wt. % of the second emulsifier, based on the total weight of the second aqueous phase.
Aspect 21 is the method of any one of Aspects 15-20, wherein the stannous ion emulsion complex comprises: 10-50 wt. % of the O/W emulsion; and 50-90 wt. % of the second aqueous phase, based on the total weight of the stannous ion emulsion complex.
Aspect 22 is an oral care product comprising a stable stannous ion complex formed using the method of any one of Aspects 1-14.

What is claimed is:

1. A method of forming a stable stannous ion complex or an oral care product, comprising:
forming a first aqueous phase by mixing water, stannous ions, and a chelating agent;
forming an oil phase by mixing an oil and a first emulsifier;
wherein a mass ratio of the oil to the first emulsifier is 1:1 to 1:20;
combining equal parts of the first aqueous phase and the oil phase to form water in oil (W/O) emulsion;
forming a second aqueous phase by mixing water, stannous ions, and a chelating agent; and
combining the water in oil emulsion and the second aqueous phase to form a stannous ion emulsion complex;
wherein a mass ratio of the water in oil emulsion to the second aqueous phase is 1:1 to 1:10.

2. The method of claim 1, wherein the first aqueous phase comprises:
5-90 wt. % of water;
5-30 wt. % of stannous ions; and
5-60 wt. % of the chelating agent, based on a total weight of the first aqueous phase.

3. The method of claim 1, wherein the first aqueous phase further comprises less than 2.5 wt. % of a pH buffer, based on a total weight of the first aqueous phase.

4. The method of claim 1, wherein the oil phase comprises:
50-95 wt. % the oil; and
5-50 wt. % of the first emulsifier, based on a total weight of the oil phase.

5. The method of claim 1, wherein the second aqueous phase comprises:
5-90 wt. % water;
5-30 wt. % of stannous ions; and
5-60 wt. % of the chelating agent, based on a total weight of the second aqueous phase.

6. The method of claim 1, wherein the second aqueous phase further comprises less than 5 wt. % of a pH buffer; and less than 20 wt. % of a second emulsifier, based on a total weight of the second aqueous phase.

7. The method of claim 1, wherein the stannous ion emulsion complex comprises:
10-50 wt. % of the W/O emulsion; and
50-90 wt. % of the second aqueous phase, based on a total weight of the stannous ion emulsion complex.

8. The method of claim 1, wherein the stannous ions are one of stannous fluoride, stannous chloride dihydrate, stannous acetate, stannous gluconate, stannous oxalate, stannous sulfate, stannous lactate, stannous tartrate, and combinations thereof.

9. The method of claim 1, wherein the chelating agent is one of GLDA, ASDA, GLDA-4Na, ASDA-4Na, and combinations thereof.

10. The method of claim 1, wherein the first emulsifier is one of span-60, span 80, AEO-3, AEO-5, AEO-7, PEG-20, PEG-40, PEG-60, PEG-80, PEG-400, tween-20, tween-60, tween-80, and combinations thereof.

11. The method of claim 1, wherein the second emulsifier is one of span-60 span 80, AEO-3, AEO-5, AEO-7, PEG-20, PEG-40, PEG-60, PEG-80, PEG-400, tween-20, tween-60, tween-80, and combinations thereof.

12. The method of claim 1, wherein the oil is at least one of α-terpineol, Eugenol, plant oil including coconut oil, soybean oil, corn oil, olive oil vitamin E oil, vitamin C, and combinations thereof.

13. The method of claim 1, further comprising dispersing the stannous ion emulsion complex in a solution to form a W/O/W stannous ion composition; and
wherein the W/O/W stannous ion composition has a Sn²⁺ stability of above 90% after a 1-month aging test in a 50° C. oven as determine by iodine titration testing.

14. An oral care product comprising a stable stannous ion complex formed using the method of claim 1.

15. A method of preparing an oral care product containing stannous ions, comprising:
forming a first aqueous phase by mixing water, stannous ions, a chelating agent, and a pH buffer;
forming an oil phase by mixing an oil and a first emulsifier;
wherein a ratio of the one oil to the first emulsifier is 1:1 to 1:20;
combining equal parts of the first aqueous phase and the oil phase to form a water in oil (W/O) emulsion;
forming a second aqueous phase by mixing water, stannous ions, a chelating agent, a pH buffer, and a second emulsifier;
combining the water in oil emulsion and the second aqueous phase to form a stannous ion emulsion complex;
wherein a ratio of the water in oil emulsion to the second aqueous phase is 1:1 to 1:10;
dispersing the stannous ion emulsion complex in a solution to form a W/O/W stannous ion composition; and
combining the W/O/W stannous ion composition with an oral care product.

16. The method of claim 15, wherein the oral care product is one of toothpaste and mouthwash; and
wherein the oral care product has an antibacterial efficacy of at least 90-99% as determined by WS/T 650-2019 antibacterial test.

17. The method of claim 15, wherein the first aqueous phase comprises:
5-90 wt. % of water;
5-30 wt. % of stannous ions;
5-60 wt. % of the chelating agent; and
less than 5 wt. % of pH buffer, based on the total weight of the first aqueous phase.

18. The method of claim 15, wherein the oil phase comprises:
50-95 wt. % the oil; and
5-50 wt. % of the first emulsifier, based on the total weight of the oil phase.

19. The method of claim 15, wherein the second aqueous phase comprises:
15-90 wt. % of water;
5-20 wt. % of stannous ions;
5-40 wt. % of the chelating agent;
less than 5 wt. % of pH buffer; and
less than 20 wt. % of the second emulsifier, based on the total weight of the second aqueous phase.

20. The method of claim 15, wherein the stannous ion emulsion complex comprises:
10-50 wt. % of the O/W emulsion; and
50-90 wt. % of the second aqueous phase, based on the total weight of the stannous ion emulsion complex.
